(19) United States
(12) Patent Application Publication
(10) Pub. No.: US 2024/0277922 A1
(43) Pub. Date: Aug. 22, 2024

(54) MEDICAL FLUID INJECTOR

(71) Applicant: INAMI Space Laboratory Inc., Tokyo (JP)

(72) Inventors: Noriaki INAMI, Tokyo (JP); Reo TAKIZAWA, Chiba (JP)

(73) Assignee: INAMI Space Laboratory Inc., Tokyo (JP)

(21) Appl. No.: 18/516,843

(22) Filed: Nov. 21, 2023

(30) Foreign Application Priority Data
Nov. 22, 2022 (JP) 2022-186186

(51) Int. Cl. A61M 5/142; A61M 5/14; A61M 5/158

(52) U.S. Cl. CPC A61M 5/14244; A61M 5/1413; A61M 5/158; A61M 2005/1401; A61M 2005/1586; A61M 2005/1588; A61M 2205/0216; A61M 2205/3334; A61M 2209/088

(57) ABSTRACT

A medical fluid injector S100 includes a medical fluid bag holder 10 that holds a medical fluid bag B containing a medical fluid, a mounting supporter 20 that supports the medical fluid bag holder 10 and in which is formed an attachment portion 22 for attaching the medical fluid bag holder 10 to a part of the body of the subject, and an elastic member 30 that is a pressurizing means for pushing out the medical fluid from the medical fluid bag B by applying pressure to the medical fluid bag B.

### CROSS-REFERENCE TO RELATED APPLICATIONS

The present application claims priority to Japanese Patent Application number 2022-186186, filed on Nov. 22, 2022, contents of which are incorporated herein by reference in their entirety.

### BACKGROUND

The present disclosure relates to a medical fluid injector. Japanese Patent Application Laid-Open No. H8-257120 discloses a medical fluid injection pump that pressurizes a medical fluid bag with a biasing force of a spring to inject a medical fluid into a subject.

In the conventional technology, since the medical fluid injection pump can be carried, there is an advantage that the subject can move during the injection of the medical fluid. However, since the position of the medical fluid injection pump is not fixed, an unexpected force may be applied to the medical fluid tube, and normal medical fluid injection may not be possible. For example, when a medical fluid is injected in a space, the medical fluid injection pump floats in the air, and such a problem tends to become apparent.

### SUMMARY

Accordingly, it is an object of the present disclosure to provide a medical fluid injector which can inject a medical fluid stably over a long period of time with a simple configuration.

According to an aspect of the present disclosure, provided is a medical fluid injector that includes a medical fluid bag holder that holds a medical fluid bag B containing a medical fluid; a mounting supporter that supports the medical fluid bag holder and in which is formed an attachment portion for attaching the medical fluid bag holder to a part of the body of the subject; and an elastic member that is a pressurizing means for pushing out the medical fluid from the medical fluid bag by applying pressure to the medical fluid bag.

### BRIEF DESCRIPTION OF THE DRAWINGS

FIG. 1 shows a medical fluid injector according to an embodiment of the present disclosure;

FIG. 2 shows a state in which the medical fluid injector is attached to a body of a subject.

FIG. 3 is a perspective view showing a state in which a medical fluid bag is attached to the medical fluid injector;

FIG. 4A is a schematic view showing a configuration of an elastic member.

FIG. 4B is a schematic view for explaining another configuration of the elastic member.

FIG. 5 shows a part of a puncture pad viewed from the inside.

FIG. 6 shows a state in which the medical fluid bag is compressed;

FIG. 7 is a schematic view showing one modification of the present disclosure.

FIG. 8 is a schematic view showing a further modification of the present disclosure.

### DESCRIPTION OF THE EMBODIMENTS

Hereinafter, the present disclosure will be described through exemplary embodiments of the present disclosure, but the following exemplary embodiments do not limit the disclosure according to the claims, and not all of the combinations of features described in the exemplary embodiments are necessarily essential to the solution means of the disclosure.

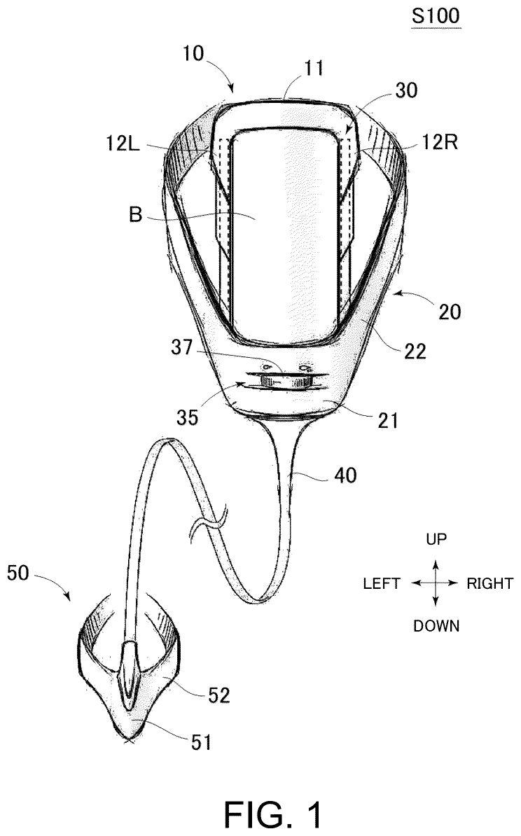

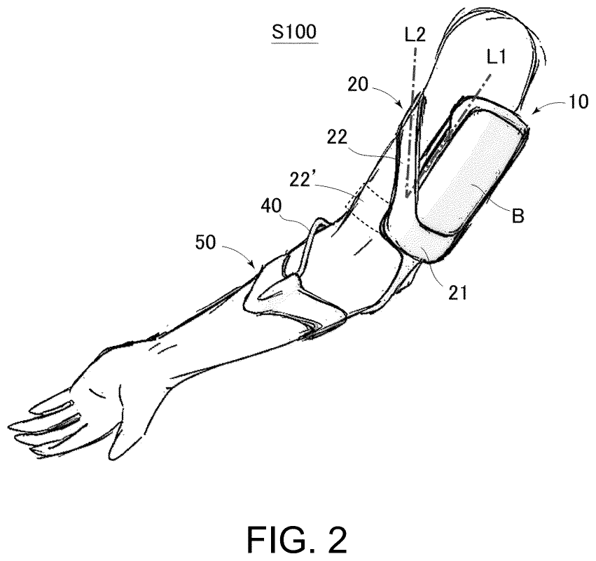

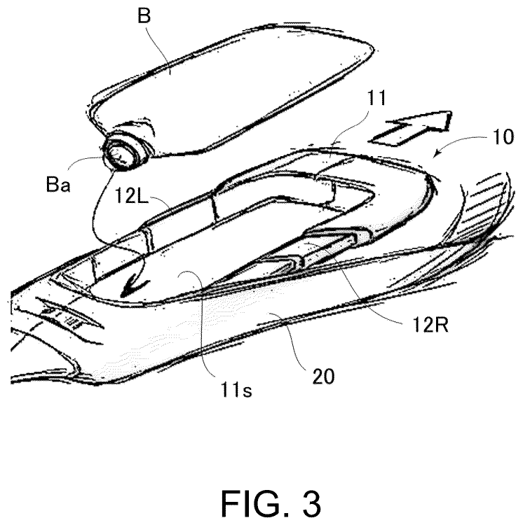

FIG. 1 shows a medical fluid injector according to an embodiment of the present disclosure. FIG. 2 shows a state in which a medical fluid injector is attached to the body of a subject. FIG. 3 is a perspective view showing a state in which a medical fluid bag is attached to the medical fluid injector;

As shown in FIG. 1, the medical fluid injector S100 includes a medical fluid bag holder 10, a mounting supporter 20, an elastic member 30, a medical fluid tube 40, and a puncture pad 50. With the medical fluid bag B being set, as shown in FIG. 2, the medical fluid injector S100 is attached to the arm of a subject, for example, and is used.

One of the features of the medical fluid injector S100 of the present embodiment is that a structure in which a medical fluid can be injected by pressurizing the medical fluid bag B with the elastic force of the elastic member 30 can be used by being attached to the body of the subject. According to this configuration, the medical fluid can be injected with a simple configuration. Moreover, the mechanism for pushing out the medical fluid is not disposed at a position away from the body of the subject. Therefore, as compared with a configuration in which the medical fluid is injected into the subject via a medical fluid tube from an apparatus at a position away from the body of the subject, it is less likely that an unexpected force would be applied to the medical fluid tube such that the medical fluid tube is bent to make it impossible to inject the medical fluid. As a result, the medical fluid can be stably injected for a long time.

### (Medical Fluid Bag B)

The medical fluid bag B contains the medical fluid to be injected into the subject. The medical fluid bag B is a bag formed of a flexible material, and as shown in FIG. 3, the medical fluid stored therein is delivered to the outside through an open tip portion Ba. The medical fluid stored in the medical fluid bag B is not limited to a specific medical fluid, and may be any medical fluid. The medical fluid may be, for example, a drug used to treat a disease, a nutritional agent, or the like.

The medical fluid bag B may have any contour shape, but has a quadrangular contour shape as an example in the present embodiment. Specifically, the medical fluid bag B has a rectangular contour shape, and in FIG. 1, the medical fluid bag B extends in a predetermined length direction (vertical direction in FIG. 1).

### (Medical Fluid Holder 10)

The medical fluid bag holder 10 is a structural portion for holding the medical fluid bag B. The medical fluid bag B is replaceably attached to the medical fluid bag holder 10. The medical fluid bag holder 10 may have any shape as long as it can hold the medical fluid bag B, but in the present embodiment, the medical fluid bag holder 10 is shaped as a frame holding the outer peripheral portion of the medical fluid bag B.

Specifically, as shown in FIGS. 1 and 3, the medical fluid bag holder 10 includes a rear end support portion 11, an extendable portion 12L, and an extendable portion 12R. The rear end support portion 11 extends so as to face the rear end of the medical fluid bag B. The extendable portion 12L and the extendable portion 12R are portions that extend so as to be bent from respective end portions of the rear end support portion 11, and extend along respective sides of the medical fluid bag B. As shown in FIG. 3, the medical fluid bag B is disposed in a space 11s surrounded by the rear end support portion 11, the extendable portion 12L, and the extendable portion 12R.

Although a member is not provided below the space 11s in FIG. 3, a support plate (not shown) for supporting the lower surface of the medical fluid bag B may be provided at this position. The structure in which the extendable portion 12L and the extendable portion 12R extend and retract will be described later together with the description of the elastic member 30.

(Mounting supporter 20)

As shown in FIG. 1, the mounting supporter 20 supports the medical fluid bag holder 10 and the elastic member 30. As shown in FIG. 2, the mounting supporter 20 has a shape capable of being attached to a part of the body of the subject. Specifically, in the present embodiment, the mounting supporter 20 has a ring shape so as to be attached to the arm of the subject.

As shown in FIG. 1, the mounting supporter 20 includes a base portion 21, an attachment portion 22, and a flow rate adjusting mechanism 35. The mounting supporter 20 is, for example, a resin component in which the base portion 21 and the attachment portion 22 are formed integrally.

The base portion 21 is a portion that covers a connection portion (not shown) between the open tip portion Ba of the medical fluid bag B and the medical fluid tube 40. The base portion 21 is connected to the end portions of the extendable portion 12L and the extendable portion 12R of the medical fluid bag holder 10. Specifically, the base portion 21 extends in a direction orthogonal to the direction in which the medical fluid bag B contracts, for example.

The attachment portion 22 has a ring shape with a size large enough to surround the arm of the subject. The attachment portion 22 is a portion for attaching the medical fluid bag holder 10 or the like to a part of the body of the subject. Instead of having a shape (see also the shape denoted by reference numeral 22′ in FIG. 2) in which a member extends as-is in the direction in which the base portion 21 extends (the left-right direction in FIG. 1), the attachment portion 22 is configured as follows. The attachment portion 22 is inclined obliquely from the base portion 21 so as to intersect with the extension direction of the base portion 21, and has a shape extending along the length direction of the arm of the subject and surrounding the arm of the subject. In other words, the attachment portion 22 is inclined in a direction L2 (specifically, a direction intersecting an acute angle) that intersects the extension direction L1 of the extendable portion. According to such a configuration, as compared with a configuration in which the attachment portion extends in a direction orthogonal to the extending direction L1 as denoted by reference numeral 22′, the medical fluid bag holder 10 can be stably held by the arm of the subject.

The mounting supporter 20 may have a C shape instead of a closed ring shape. Specifically, the overall shape of the mounting supporter 20 may be formed into a C shape by extending the extension portion surrounding the arm of the subject from each of the left end portion and the right end portion of the base portion 21.

### (Elastic Member 30)

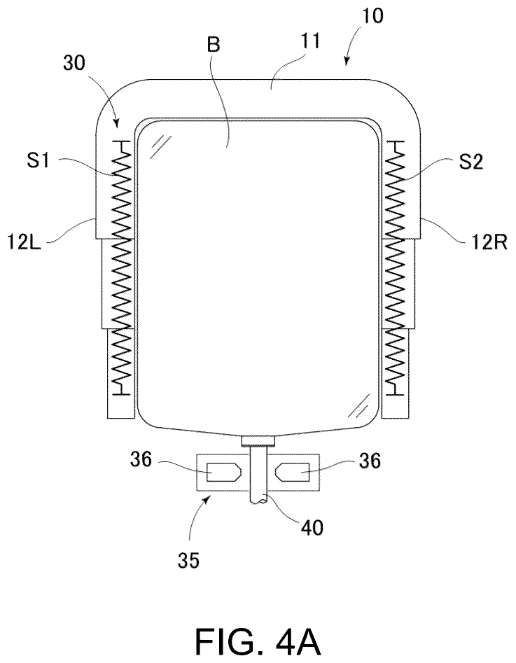

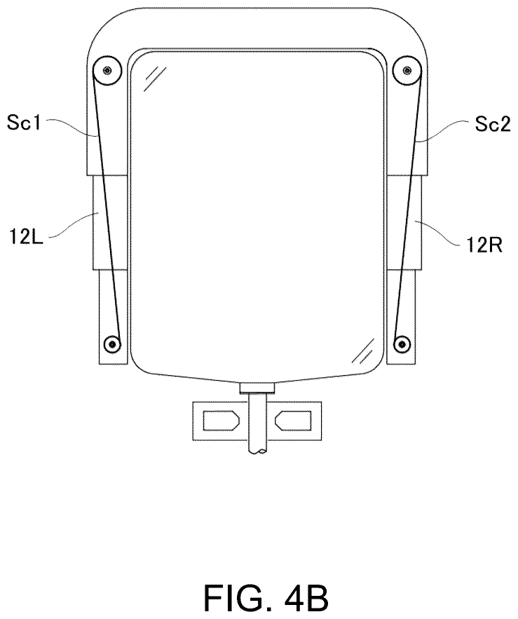

FIG. 4A is a schematic view showing a configuration of the elastic member 30. The elastic member 30 is a pressurizing means for pushing out the medical fluid by applying pressure to the medical fluid bag B. In the present embodiment, a spring S1 and a spring S2 are provided as the elastic member 30. The springs S1 and S2 are, for example, coil springs. The spring S1 is disposed in the extendable portion 12L of the medical fluid bag holding section 10, and the spring S2 is disposed in the extendable portion 12R.

The extendable portion 12L and the extendable portion 12R have a hollow structure in which a plurality of members are connected to each other in the length direction of the members, and are extendable. The spring S1 and the spring S2 are disposed inside the extendable portion 12L and the extendable portion 12R, respectively.

As shown in FIG. 4A, in a state in which the extendable portion 12L and the extendable portion 12R are extended, the springs S1 and S2 are extended, and as a result, a force is applied to the rear end support portion 11 in a direction causing the rear end support portion 11 to be moved toward the base portion 21 (FIG. 1). In this way, in the medical fluid injector S100 of the present embodiment, the rear end support portion 11 is moved by the action of the elastic member 30, and a force is applied to the medical fluid bag B in a direction causing the medical fluid bag B to contract in the length direction. Due to this, the medical fluid is pushed out from the medical fluid bag B.

The elastic member 30 is not limited to a coil spring, and may be a constant load spring. The constant load spring generates a constant load regardless of the extension distance. In a case where the elastic member 30 is a constant load spring, a constant force is applied to the rear end support portion 11 without being affected by the extension and contraction state of the elastic member 30. Therefore, the medical fluid can be pushed out at a constant flow rate regardless of the remaining amount of medical fluid in the medical fluid bag B.

FIG. 4B is a schematic view for explaining another configuration of the elastic member. In this example, a constant load spring Sc1 and a constant load spring Sc2 are disposed inside the extendable portion 12L and the extendable portion 12R, respectively. In this way, a first roll of the constant load spring may be disposed at one end of the extendable portion, a second roll may be disposed at the other end of the extendable portion, and the constant load spring may be disposed so that the plate-shaped spring body extends between these rolls.

In the configuration of FIGS. 1 to 4B, by moving the rear end support portion 11 toward the side away from the base portion 21 to extend the springs S1 and S2, a force is generated in a direction causing the medical fluid bag B to be pressed and contract, thereby making it possible for the medical fluid to be injected.

### (Flow Rate Adjusting Mechanism 35)

The flow rate adjusting mechanism 35 is a mechanism for adjusting the flow rate of the medical fluid delivered toward the subject. As shown in FIG. 4A, the flow rate adjusting mechanism 35 includes a pair of clamp members 36 and an adjustment dial 37 (FIG. 1).

The pair of clamp members 36 sandwich the medical fluid tube 40 connected to the medical fluid bag B from the outside. The distance between the clamp members 36 is changed by rotating the adjustment dial 37. Specifically, the adjustment dial 37 and the clamp members 36 are mechanically connected by a transmission mechanism (not shown), for example, and when the adjustment dial 37 is rotated, a force is transmitted to the clamp members 36 by the transmission mechanism. Due to this, the clamp members 36 move.

A part of the adjustment dial 37 is provided so as to project to the outside of the mounting supporter 20, so that the subject can turn the adjustment dial 37. By operating the adjustment dial 37, the pair of clamp members 36 are moved, and the medical fluid tube 40 is squeezed to change the cross-sectional area of the flow path inside the medical fluid tube 40. In this way, the flow rate delivered toward the subject is adjusted.

The flow rate adjusting mechanism 35 may not only adjust the flow rate but also switch between a state in which the medical fluid is injected and a state in which the medical fluid is not injected. Alternatively, although not shown, a switching mechanism for switching between a state in which the medical fluid is injected and a state in which the medical fluid is not injected may be provided separately from the flow rate adjusting mechanism 35.

### (Puncturing Pad 50)

As shown in FIG. 1, the puncture pad 50 includes a base portion 51 and an attachment portion 52.

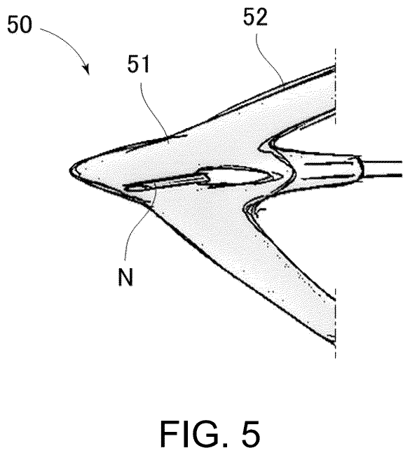

FIG. 5 shows a part of the puncture pad 50 viewed from the inside. The base portion 51 supports an injection needle N in a replaceable manner. The base portion 51 may be formed of a transparent material, for example, so that the needle tip can be easily recognized when the injection needle N is inserted.

The attachment portion 52 is a member for fixing the base portion 51 to a predetermined position on the body of the subject. For example, the attachment portion 52 is formed in a ring shape or a C shape. The shape of the attachment portion 52 is not necessarily limited, as long as the attachment portion 52 can fix the base portion 51 to a part of the body of the subject.

### (Usage Method)

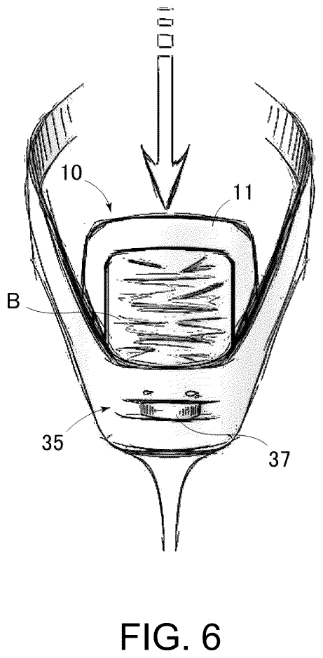

A method of using the medical fluid injector S100 configured as described above will be described. FIG. 6 shows a state in which the medical fluid bag is compressed. First, a person who uses the medical fluid injector S100 attaches the medical fluid bag holder 10 to his/her arm, in a state where the medical fluid bag B is attached, as shown in FIG. 2.

In a case where the flow rate adjusting mechanism 35 has a function of switching between a state in which the medical fluid is injected and a state in which the medical fluid is not injected, the subject operates the adjustment dial 37 of the flow rate adjusting mechanism 35 to start the injection of the medical fluid in a state where the distal end of the injection needle N is inserted into a blood vessel.

The rear end support portion 11 is moved by the elastic force of the elastic member 30, whereby pressure is applied to the medical fluid bag B. As a result, the medical fluid in the medical fluid bag B is injected into the body of the subject through the medical fluid tube 40 and the injection needle N. As shown in FIG. 6, when the medical fluid bag B is pressed by the rear end support portion 11 of the medical fluid bag holder 10, the medical fluid bag B is gradually compressed along the length direction of the medical fluid bag B. The injection of the medical fluid ends when the subject returns the adjustment dial 37 to a predetermined position at which the medical fluid is not injected, for example.

### Effect of the Configuration of the Present Embodiment

As described above, according to the medical fluid injector S100 of the present embodiment, since the medical fluid bag B is pressurized by using the elastic force of the elastic member 30, the medical fluid can be injected with a simple configuration. Moreover, since the mechanism for pushing out the medical fluid is not disposed at a position away from the body of the subject, but is instead attached to a part of the body as shown in FIG. 2, a problem that the medical fluid tube 40 connecting the mechanism for pushing out the medical fluid and the injection needle N becomes bent and the medical fluid cannot be injected is less likely to occur. Therefore, according to the medical fluid injector S100, the medical fluid can be injected stably over a long period of time.

The medical fluid injector S100 can also be used in space. In space, for example, even if a medical fluid pump is provided so as to be portable, the medical fluid pump cannot be placed near the subject such as it is on the earth. On the other hand, an apparatus capable of attaching the medical fluid bag B and the mechanism for pushing out the medical fluid to the subject as in the present embodiment is particularly suitable for use in space.

Further, in the present embodiment, a force is applied in a direction causing the medical fluid bag B to contract in the length direction, and the medical fluid bag B is deformed so as to be compressed in the length direction. In the present disclosure, the medical fluid bag B may be pressed in the thickness direction, but according to the configuration shown in FIG. 1, since the number of components arranged in the thickness direction of the medical fluid bag B can be reduced, for example, there is an advantage that the medical fluid bag B is not bulky when the medical fluid injector S100 is attached to the body of the subject as shown in FIG. 2.

In the present embodiment, the spring S1 and the spring S2 are disposed inside the extendable portion 12L and the extendable portion 12R having a hollow structure. According to such a configuration, an imbalance is less likely to occur in the force for pulling the rear end support portion 11 in the left and right extendable portions, and the medical fluid bag B can be pressurized with good balance. Further, since the spring S1 and the spring S2 are not exposed to the outside, safety for the subject is enhanced. Further, the medical fluid is prevented from adhering to the spring S1 and the spring S2.

Further, in the present embodiment, the flow rate adjusting mechanism 35 is provided in a part of the mounting supporter 20. Therefore, as compared with a configuration in which a mechanism for adjusting the flow rate is disposed in the middle of the medical fluid tube 40 extending from the mounting supporter 20, the configuration is simplified (i.e., the number of elements formed separately is reduced), and handling properties are improved.

Further, in the present embodiment, a ring-shaped or C-shaped attachment portion 22 is provided on the mounting supporter 20 so as to be attached to the arm of the subject. According to such a configuration, the structure including the medical fluid bag holder 10, the elastic member 30, and the like, to which the medical fluid bag B is mounted, can be easily attached to the body as compared with a method of attaching the structure to the body using, for example, a band, a belt, or the like.

### <Modification>

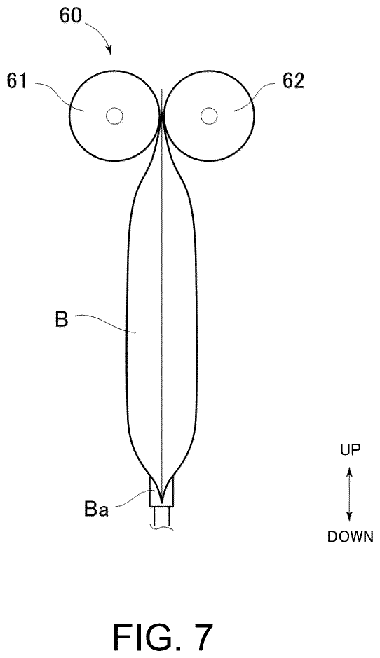

The present disclosure is not limited to the specific embodiments described above, and can be modified in various ways. FIG. 7 is a schematic diagram showing one modification of the present disclosure. As shown in FIG. 7, a pressurizing means 60 for applying pressure to the medical fluid bag B may include a roller 61 and a roller 62. For example, the pressurizing means 60 may include an elastic member (not shown) for moving the roller 61 and the roller 62 toward the open tip portion Ba. As in the embodiment described above, the elastic member may be a coil spring, a ridge, a constant load spring, or the like.

As another example, the pressurizing means 60 may include a drive mechanism (not shown) for rotating one or both of the rollers 61 and 62. The drive mechanism may include, for example, a motor.

Instead of using a roller such as shown in FIG. 7, a mechanism for pressurizing the medical fluid bag B from the outside with the pressure of air may be provided as a pressurizing unit, for example. Such a pressurizing means may compress the medical fluid bag B in the thickness direction or in the length direction.

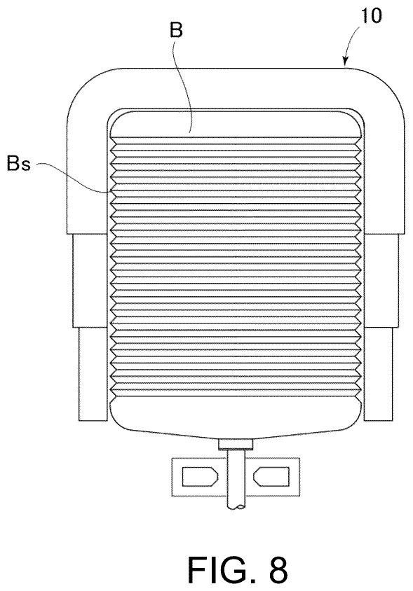

FIG. 8 is a schematic diagram showing a further modification of the present disclosure. As shown in FIG. 8, the medical fluid bag B may be formed in a bellows shape. Specifically, a bellows portion Bs is formed in the medical fluid bag B in order to expand and contract the medical fluid bag B. According to such a configuration, since the medical fluid bag B is likely to contract in the length direction, the medical fluid can be suitably injected into the subject.

Although the embodiments of the present disclosure have been described above, the technical scope of the present disclosure is not limited to the embodiments described above, and various modifications and changes can be made without departing from the scope of the present disclosure. For example, all or a portion of the device may be functionally or physically distributed and integrated in arbitrary units. Further, new embodiments resulting from arbitrary combinations of a plurality of embodiments are also included in the embodiments of the present disclosure. The effect of the new embodiment caused by the combination has the effect of the original embodiment.

### DESCRIPTION OF REFERENCE NUMERALS

10 fluid bag holder
11 rear end support portion
11s space
12L extendable portion
12R extendable portion
20 mounting supporter
21 base portion
22 attachment portion
30 elastic member
35 flow rate adjusting mechanism
36 clamp member
37 adjustment dial
40 medical fluid tube
50 puncture pad
51 base portion
52 attachment portion
60 pressurizing means
61 roller
62 roller
B medical fluid bag
Ba open tip portion
N injection needle
S1, S2 spring
Sc1, Sc2 constant load spring
S100 medical fluid injector

1. A medical fluid injector comprising:
a medical fluid bag holder that holds a medical fluid bag containing a medical fluid;
a mounting supporter that supports the medical fluid bag holder and in which is formed an attachment portion for attaching the medical fluid bag holder to a part of a body of a subject; and
a pressurizing means for pushing out the medical fluid from the medical fluid bag by applying pressure to the medical fluid bag.

2. The medical fluid injector according to claim 1, wherein
the medical fluid bag extends in a predetermined length direction, and
the pressurizing means is an elastic member that applies a force in a direction in which the medical fluid bag contracts in the length direction.

3. The medical fluid injector according to claim 2, wherein
the medical fluid bag holder includes a plurality of extendable portions having hollow structures extending along respective sides of the medical fluid bag, and
the elastic member is disposed inside each of the extendable portions.

4. The medical fluid injector according to claim 3, wherein
the elastic member is a constant load spring disposed inside the extendable portion.

5. The medical fluid injector according to claim 1, wherein
the mounting supporter further includes a flow rate adjusting mechanism that sandwiches a medical fluid tube connected to the medical fluid bag from the outside and changes a cross-sectional area of a flow path inside the medical fluid tube.

6. The medical fluid injector according to claim 1, wherein
the attachment portion has a ring shape or C shape so as to be attached to an arm of the subject.

7. The medical fluid injector according to claim 6, wherein
the medical fluid bag holder includes a plurality of extendable portions having a hollow structure extending along respective sides of the medical fluid bag, and
the attachment portion extends in a direction intersecting with an extension direction of the extendable portions.
